(19) United States
(12) Patent Application Publication
(10) Pub. No.: US 2022/0196524 A1
(43) Pub. Date: Jun. 23, 2022

(54) PROCESS FOR PREPARING A PEPTIDE SAMPLE

(71) Applicants: CENTRE HOSPITALIER UNIVERSITAIRE DE MONTPELLIER, Montpellier (FR); UNIVERSITE DE MONTPELLIER, Montpellier (FR); SPOT TO LAB, Montpellier Cedex 5 (FR)

(72) Inventors: Sylvain LEHMANN, Castelnau-le-Lez (FR); Christophe HIRTZ, Montpellier (FR); Jérôme VIALARET, Saint-Paul-et-Valmalle (FR)

(21) Appl. No.: 17/595,460

(22) PCT Filed: May 19, 2020
(86) PCT No.: PCT/EP2020/063944

§ 371 Date: Nov. 17, 2021

(30) Foreign Application Priority Data
May 20, 2019 (FR) 1905247

(51) Int. Cl. G01N 1/40; G01N 1/34; G01N 33/68

(52) U.S. Cl. CPC G01N 1/4044; G01N 33/6848; G01N 1/34; G01N 1/4005

(57) ABSTRACT

A process for preparing a peptide sample from a biological sample, and a process for detecting or quantifying proteins including the process for preparing a sample. Also disclosed is the use of these processes for the detection or monitoring of a condition or a disease.

### FIELD OF THE INVENTION

The present application relates to a process for preparing a peptide sample from a biological sample. It also relates to a process for detecting and quantifying proteins implementing said process for preparing the peptide sample. Finally, it relates to the use of these processes for the detection or monitoring of a condition or a disease.

The technical field of the invention is that of the biochemical analysis of proteins originating from a biological sample.

### STATE OF THE ART

The detection and monitoring of the progression of particular diseases or physical conditions, such as deficiencies or malnutrition, require the detection and quantification of specific proteins, called “clinical protein markers”, which are essential tools for prevention, diagnosis, prognosis and treatment choices.

The detection of clinical protein markers in a complex biological sample requires, for a proteomic or mass spectrometry analysis, a first step of preparing a peptide sample, the quality of which is vital for then allowing a sensitive and reproducible analysis. In fact, the analysis of these media is subject to interference due to the presence of proteins in a large majority and, in certain cases, to the release of intracellular contents during the preparation of the sample. This is the case in particular for blood products in which haemoglobin, albumin or immunoglobulins are very abundant.

Conventional methods for analysing blood products apply to products in liquid or solid form, such as for example dried blood on blotting paper, the latter form in fact having a number of practical, analytical, clinical and financial advantages. Methods for extracting small non-protein molecules (amino acids, steroid hormones, vitamins and medicaments) from solid samples are already known and validated. In contrast, the analysis of clinical protein markers encounters a number of analysis problems as a result of the interference due to the other proteins in a large majority, the low efficiency and reproducibility of the methods for preparing or digesting proteins and the possible presence of impurities in the solid support. Recent advances in mass spectrometry make an interesting alternative to conventional clinical analysis processes that have already been validated (immunodetection, etc.) possible. The difference in protein composition between a solid blood sample and a serum or plasma sample, as well as the complexity of these matrices, currently means that analysis by mass spectrometry is not very reproducible, not very sensitive and not very compatible with an acceptable clinical use.

There is therefore a significant need to have a reproducible, sensitive, fast and clinically validated process for preparing peptide samples prior to the detection and analytical monitoring of clinical protein markers, in particular by mass spectrometry.

Processes for preparing peptide samples prior to an analysis are described in the prior art. These processes comprise steps of denaturation, elution, alkylation and trypsin digestion of the proteins.

WO 2014/118474 describes a process for determining the presence of prolidase for the detection of colorectal cancer; this process comprises a step of enzymatic digestion followed by a step of cleaning of the proteins on an HLB solid-phase support from Waters.

Yakundi et al. (Journal of Pharmaceutical and Biomedical analysis, 56, 1057-1063, 2011) mentions a process intended to quantify ranitidine in a dried blood sample, for a clinical application. This process comprises a step of cleaning of the proteins prior to an analysis by mass spectrometry, and does not comprise a step of enzymatic digestion.

Rosting et al. (American Chemical Society, 87, 7918-24, 2015) discloses a process for detecting a model protein in a dried blood sample, comprising a step of enzymatic digestion followed by a pre-concentration by solid-phase extraction (SPE), before analysis by mass spectrometry.

### DESCRIPTION OF THE INVENTION

The inventors have now developed a process for the in vitro preparation of a peptide sample, this process comprising, in succession, steps of denaturation, reduction/alkylation, cleaning of the proteins by chromatography on a solid support comprising at least one polystyrene-divinylbenzene polymer, then enzymatic digestion.

A process according to the invention makes the reproducible detection of a large number of clinical marker proteins from a low-volume sample possible, this process can be successfully applied to a solid or liquid sample, such as plasma, and can be automated. Analyses of peptide samples by mass spectrometry have the advantage of absolute specificity in the detection of proteins and are considered to be reference methods. A preparation process according to the invention, followed by an analysis using a process such as mass spectrometry for example, makes it possible to have results that are statistically comparable to clinically validated tests, such as for example immunoassays.

In comparison with the processes of the state of the art, a process according to the invention makes it possible to eliminate the interference due to the release of intracellular contents and to the presence of proteins in a majority, thus making it possible to improve the sensitivity, specificity and analytical performance of the analysis of the compounds.

A process for preparing a peptide sample according to the invention is suitable for the analysis of any type of biological sample, in particular blood samples, and more particularly solid blood samples, for which no reproducible and clinically validated process is yet available. The use of a solid support of the blotting paper type is a recognized method of blood collection because it is non-invasive (taken from a fingertip), easy to transport (in particular by post) and easy to implement; it can be carried out by the subject themselves or by an unqualified person and is not dangerous because the sample is decontaminated by drying. A process according to the invention implemented on a sample on a solid support makes a low-cost analysis possible, in particular for longitudinal monitoring of subjects for multiple markers. Such an approach is promising for monitoring populations for whom access to the analysis laboratories is restricted and for future developments in telemedicine.

According to a first subject, the present application relates to a process for the in vitro preparation of a peptide sample from a biological sample, comprising the following successive steps: a) denaturation of the proteins present in said sample, b) reduction and alkylation of the proteins resulting from step a), c) cleaning of the proteins resulting from step b) by reversed-phase chromatography on a solid polymeric support, said solid support comprising at least one polystyrene-divinylbenzene polymer, and d) digestion of the proteins resulting from step c) by a protease.

A process according to the invention comprises at least one step of cleaning of the denatured, reduced and alkylated proteins and a step of enzymatic digestion, said step of cleaning of the proteins taking place prior to the step of enzymatic digestion of said proteins.

By “biological sample” is meant a sample comprising tissues and cells originating from the body, human or animal, and their derivatives, and in particular the following products: blood, serum, plasma, urine, faeces, saliva, sputum, biopsies, and any secretions and body fluids.

According to a first particular aspect of the process according to the invention, the biological sample is a blood product in liquid form, selected from: whole blood, serum and plasma.

According to a second particular aspect of the process according to the invention, the biological sample is a blood product in solid, or dried, form selected from whole blood, serum and plasma. According to an even more particular aspect, the biological sample is constituted by dried whole blood deposited on a solid support, also called Dried Blood Spot (DBS).

Typically, taking such a sample consists of depositing a drop of blood with a volume of from 30 to 50 μL on a suitable solid support followed by a drying step.

The solid support of the sample is then easy to handle and can in particular be sent by standard post.

Of the suitable solid supports there may be mentioned: blotting or collection papers such as “Whatman 903 paper” or “Ahlstrom TFN/226 paper”, on which blood can be deposited.

By “polystyrene-divinylbenzene polymer” is meant a polymer comprising polystyrene cross-linked by divinylbenzene. Polystyrene, or poly(1-phenylethylene), is obtained by polymerization of the styrene monomer. Divinylbenzene, or DVB, is an aromatic hydrocarbon of formula C₁₀H₁₀ used as cross-linking agent.

In a process according to the invention, a solid support for cleaning proteins comprises at least one polystyrene-divinylbenzene polymer possibly combined with another polymer or with any other suitable element.

The solid support in the reversed-phase chromatography step is characterized by the presence of polystyrene-divinylbenzene polymer beads the size of which is comprised between 10 and 50 mm, preferably between 20 and 40 mm and preferably 30 mm, on the one hand, and pores the size of which is comprised between 500 and 10,000 Angstrom, and preferably between 2000 and 4000 Angstrom, on the other hand.

Of the solid supports comprising at least one polystyrene-divinylbenzene polymer that can be used in a process according to the invention, there may be mentioned, in particular, the RP-W® cartridges marketed by Agilent.

Said RP-W® cartridges are described for a use in a process and an application that is very different from that of the process according to the invention. In fact, the processes of the state of the art typically describe the use of these cartridges during the production of recombinant antibodies, for a “desalting” step, i.e. in particular to ensure the elimination of the 6 M guanidine that is necessary for the denaturation of the antibodies, prior to the digestion of the latter by trypsin.

In a process according to the invention, each of the steps of denaturation, reduction, alkylation and enzymatic digestion of the proteins is carried out by means of any process well known to a person skilled in the art, under suitable buffer, concentration, temperature and duration conditions. Preferably, the denaturation is carried out by virtue of the addition of an agent that denatures the proteins, for example 8 M urea, the reduction is carried out by virtue of the addition of an agent that is capable of reducing the disulfide bonds of the proteins, for example DTT, the alkylation of the cysteines released during the reduction is carried out by virtue of the addition of an alkylating agent, for example iodoacetic acid (IAA).

The enzymatic digestion of the proteins is preferably carried out by the addition of at least one protease selected from: trypsin, endoproteinase Glu-C and endoproteinase Lys-C. According to a particular aspect of a process according to the invention, the enzymatic digestion is carried out for a duration greater than 2 hours, preferably greater than or equal to 10 hours, preferably greater than or equal to 14 hours, preferably equal to 14 hours.

More particularly, in a process according to the invention, the enzymatic digestion is carried out in the presence of a ratio between the quantity of enzyme present and the quantity of substrate protein comprised between 1/10 and 1/200 and preferably comprised between 1/50 and 1/100. A person skilled in the art, specializing in the field, is capable of identifying and calculating said ratio precisely.

According to another particular aspect of said first subject, the present application relates to a process for the in vitro preparation of a peptide sample from a blood sample in solid or dried form, said process comprising a step of extraction of the proteins from said solid blood sample prior to the steps of denaturation, reduction and alkylation, cleaning of the proteins and digestion by a protease.

The extraction of the proteins is carried out by any extraction process known to a person skilled in the art, in particular by bringing the sample and ammonium bicarbonate together at ambient temperature, possibly in the presence of ovalbumin.

According to a second subject, the present application relates to a process for detecting the presence of at least one protein considered to be a clinical marker in a biological sample, said detection process comprising an in vitro process for preparing a peptide sample, according to the invention, followed by a step of detection of the presence of at least one protein in said sample.

According to a particular embodiment, the present application relates to a process for detecting and quantifying at least one protein in a biological sample, said detection and quantification process comprising an in vitro process for preparing a peptide sample, according to the invention, followed by a step of detection and quantification of at least one protein in said sample.

The detection and/or quantification of said at least one protein is carried out by any known technical means, preferably by mass spectrometry, more preferably by mass spectrometry combined with liquid chromatography, or Liquid Chromatography-Mass Spectrometry (LC-MS). Said detection and/or quantification step can be implemented for one, two or more proteins. Said mass spectrometry can be carried out in a multiplexed form.

The detection and quantification of proteins in a complex biological sample by LC-MS typically comprises the preparation of a peptide sample, then the liquid-phase separation by chromatography of said peptide sample, followed by the analysis by mass spectrometry, with the identification, possibly followed by the quantification, of proteins, then the statistical analysis of the results.

The detection and/or quantification of proteins is carried out by using at least one standard consisting of a labelled or non-labelled peptide, making it possible to detect the corresponding specific protein. Said standards are well known to a person skilled in the art, who can easily select them from the available commercial standards depending on the nature of the protein(s) of interest.

According to a particular embodiment, a subject of the present invention is a process for detecting, possibly followed by quantifying, at least one protein selected from the group consisting of the following proteins: afamin, alpha-1 antichymotrypsin, alpha-1B-glycoprotein (A1BG), alpha-1-acid glycoprotein, albumin (ALBU), alpha-2-HS-glycoprotein, alpha-2-antiplasmin, alpha-2-macroglobulin (A2MG), antithrombin-3 (ANT3), apolipoprotein B100 (Apo B100), apolipoprotein C2 (Apo C2), apolipoprotein D, apolipoprotein E (Apo E), apolipoprotein M, apolipoprotein (a), apolipoprotein al (Apo A1), apolipoprotein A2 (Apo A2), apolipoprotein a4, beta-2-glycoprotein 1, beta-2-microglobulin (B2M), beta-Ala-His-dipeptidase, C4b-binding protein (alpha chain), CD5 antigen-like, cDNA-F1153327, ceruloplasmin (CERU), cholinesterase, clusterin, coagulation factor X (CF-X), coagulation factor XI, coagulation factor XII, complement C1q subcomponent subunit B, subunit C of complement C1q, complement C1r subcomponent, complement C1s subcomponent, complement C2 (C2), complement C3 (C3), complement C4B (C4B), complement C5, complement component C8 (beta chain), complement component C9, complement factor B, complement factor D, complement factor I, cystatin C (CysC), corticoid-binding globulin, C-reactive protein (CRP), fibrinogen (alpha chain) (FIBA), fibrinogen (beta chain) (FIBB), fibronectin, fibulin-1, gelsolin, haptoglobin, haemoglobin subunit alpha, haemopexin, heparin cofactor 2, the acid-labile subunit of insulin-like growth factor binding protein, insulin-like growth factor binding protein 3, haptoglobin (HPT), inter-alpha-trypsin inhibitor heavy chain H1, inter-alpha-trypsin inhibitor heavy chain H2, insulin-like growth factor binding protein 3 (IGFB3), lipopolysaccharide-binding protein, lumican, neuropilin-2, orosomucoid (ORM), PEDF, plasminogen (PLMN), protein AMBP, prothrombin, retinol-binding protein 4, serotransferrin (TRANSF), serum amyloid A4 protein, thyroxine-binding globulin, transthyretin (prealbumin) (TTHY), vasorin, vitamin D-binding protein, vitamin K-dependent protein C, vitamin K-dependent protein S, retinol-binding protein 4 (RET4).

According to a more particular embodiment, a subject of the present invention is a process for detecting, possibly followed by quantifying, one, two, three, four, five, six, seven, eight, nine, ten, fifteen, twenty, twenty-five, thirty, thirty-five, forty or more than forty proteins selected from the group defined above.

According to a third subject, the present application relates to a kit suitable for the in vitro preparation of a peptide sample from a biological sample according to the invention, said kit comprising at least:
reagents suitable for the denaturation of proteins,
reagents suitable for the reduction of disulfide bonds and possibly alkylating agents,
reagents suitable for the enzymatic digestion of proteins, and
reagents suitable for reversed-phase chromatography on a solid support, said solid support comprising at least one polymer selected from the family of polystyrene-divinylbenzene polymers.

Said reagents are selected from the existing reagents, which are well known to a person skilled in the art.

According to a fourth subject, the present application relates to the use of a process for the in vitro preparation of a peptide sample from a biological sample according to the invention. According to a particular embodiment, the present application relates to the use of a process for detecting and possibly quantifying at least one protein in a biological sample, according to the invention.

According to a particular embodiment, a subject of the present invention is the use of a process for the in vitro preparation of a peptide sample from a biological sample, according to the invention, and/or the use of a detection and potentially quantification process according to the invention, for the detection and potentially quantification of at least one protein selected from the group consisting of the following proteins: afamin, alpha-1-antichymotrypsin, alpha-1B-glycoprotein (A1BG), alpha-1-acid glycoprotein, albumin (ALBU), alpha-2-HS-glycoprotein, alpha-2-antiplasmin, alpha-2-macroglobulin (A2MG), antithrombin-3 (ANT3), apolipoprotein B100 (Apo B100), apolipoprotein C2 (Apo C2), apolipoprotein D, apolipoprotein E (Apo E), apolipoprotein M, apolipoprotein (a), apolipoprotein al (Apo A1), apolipoprotein A2 (Apo A2), apolipoprotein a4, beta-2-glycoprotein 1, beta-2-microglobulin (B2M), beta-Ala-His-dipeptidase, C4b-binding protein (alpha chain), CD5 antigen-like, cDNA-F1153327, ceruloplasmin (CERU), cholinesterase, clusterin, coagulation factor X (CF-X), coagulation factor XI, coagulation factor XII, complement C1q subcomponent subunit B, subunit C of complement C1q, complement Cir subcomponent, complement Cis subcomponent, complement C2 (C2), complement C3 (C3), complement C4B (C4B), complement C5, complement component C8 (beta chain), complement component C9, complement factor B, complement factor D, complement factor I, cystatin C (CysC), corticoid-binding globulin, C-reactive protein (CRP), fibrinogen (alpha chain) (FIBA), fibrinogen (beta chain) (FIBB), fibronectin, fibulin-1, gelsolin, haptoglobin, haemoglobin subunit alpha, haemopexin, heparin cofactor 2, the acid-labile subunit of insulin-like growth factor binding protein, insulin-like growth factor binding protein 3, haptoglobin (HPT), inter-alpha-trypsin inhibitor heavy chain H1, inter-alpha-trypsin inhibitor heavy chain H2, insulin-like growth factor binding protein 3 (IGFB3), lipopolysaccharide-binding protein, lumican, neuropilin-2, orosomucoid (ORM), PEDF, plasminogen (PLMN), protein AMBP, prothrombin, retinol-binding protein 4, serotransferrin (TRANSF), serum amyloid A4 protein, thyroxine-binding globulin, transthyretin (prealbumin) (TTHY), vasorin, vitamin D-binding protein, vitamin K-dependent protein C, vitamin K-dependent protein S, retinol-binding protein 4 (RET4).

According to a more particular embodiment, a subject of the present invention is the use of a process for the in vitro preparation of a peptide sample from a biological sample, according to the invention, and/or the use of a detection and potentially quantification process according to the invention, for the detection and potentially quantification of one, two, three, four, five, six, seven, eight, nine, ten, fifteen, twenty, twenty-five, thirty, thirty-five, forty or more than forty proteins selected from the group defined above.

According to a fifth subject, the present application relates to the use of a process for the in vitro preparation of a peptide sample from a biological sample according to the invention for the detection and/or monitoring of a particular condition or disease selected from:
neurodegeneration, in particular Alzheimer's disease,
a neurological or psychiatric disease, and in particular multiple sclerosis and autism,
a state of deficiency, infection, inflammation or malnutrition,
a chronic or progressive disease, in particular cancer, hepatitis or a metabolic disease.

The detection and/or monitoring of a particular disease or condition can be carried out by detecting and/or quantifying at least one protein considered to be a clinical marker, possibly combined with the detection and/or quantification of at least one non-protein clinical marker.

According to a particular aspect, a subject of the present application is the use of a process for the in vitro preparation of a peptide sample from a biological sample, according to the invention, for the detection and/or monitoring of the state of health, nutrition and frailty of a subject, in particular an elderly subject. By elderly subject is meant a subject 75 years of age or older, more particularly a subject 85 years of age or older. More particularly, a use according to the invention comprises the detection and/or quantification of one or more proteins selected from: albumin, alpha-1-acid glycoprotein, transthyretin and CRP (C-reactive protein).

Other advantages and characteristics of the invention will become apparent on examining the detailed description of an embodiment, which is in no way limitative, and the attached drawings.

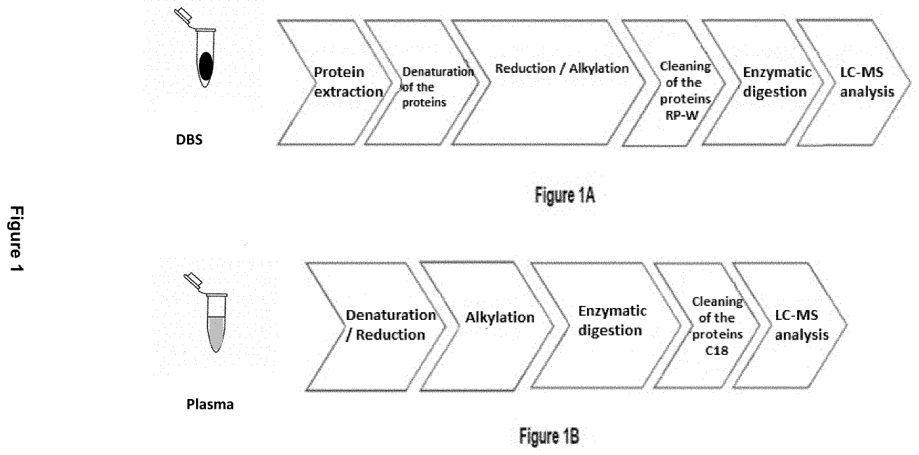

FIG. 1 diagrammatically represents an embodiment of a process for preparing a peptide sample according to the invention from a DBS sample (FIG. 1A) and an embodiment of a process for preparing a peptide sample according to the state of the art from plasma (FIG. 1B).

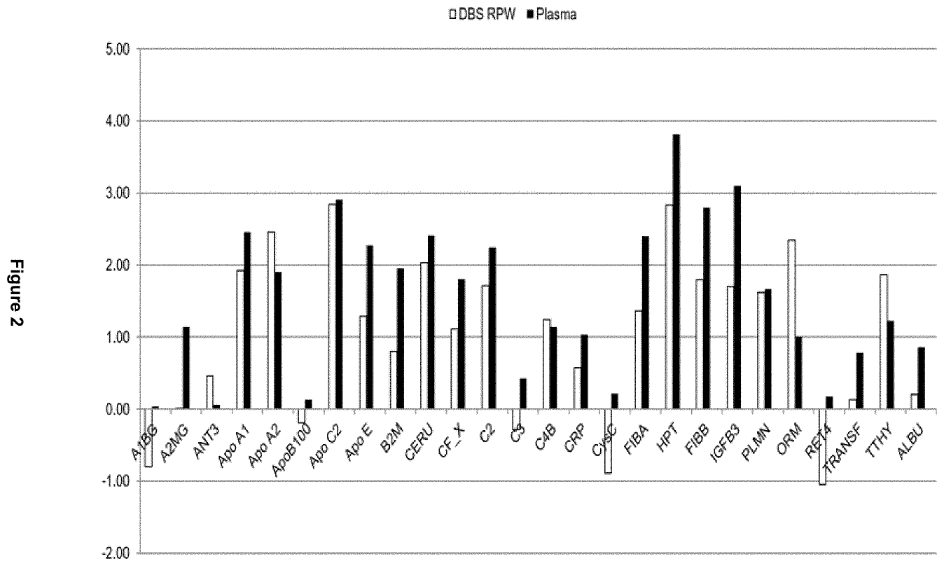

FIG. 2 represents a histogram showing the compared results of the LC-MS analysis of 26 different proteins, carried out on samples prepared according to three different protocols: a) process according to the invention applied to a dried blood sample (“DBS RPW”), b) process according to the invention applied to a plasma sample (“plasma”), c) process for preparing DBS samples according to the state of the art (“standard DBS preparation”). For each of the proteins, the values of the areas obtained during the analysis by mass spectrometry under the “DBS RPW” and “plasma” conditions are divided, respectively, by the corresponding values of the areas obtained with the standard process for preparing the sample. LOG (area/area after standard DBS preparation) is represented on the y axis, the results for each of the 26 proteins are represented on the x axis, with “DBS RPW” as a light histogram and “plasma” as a dark histogram.

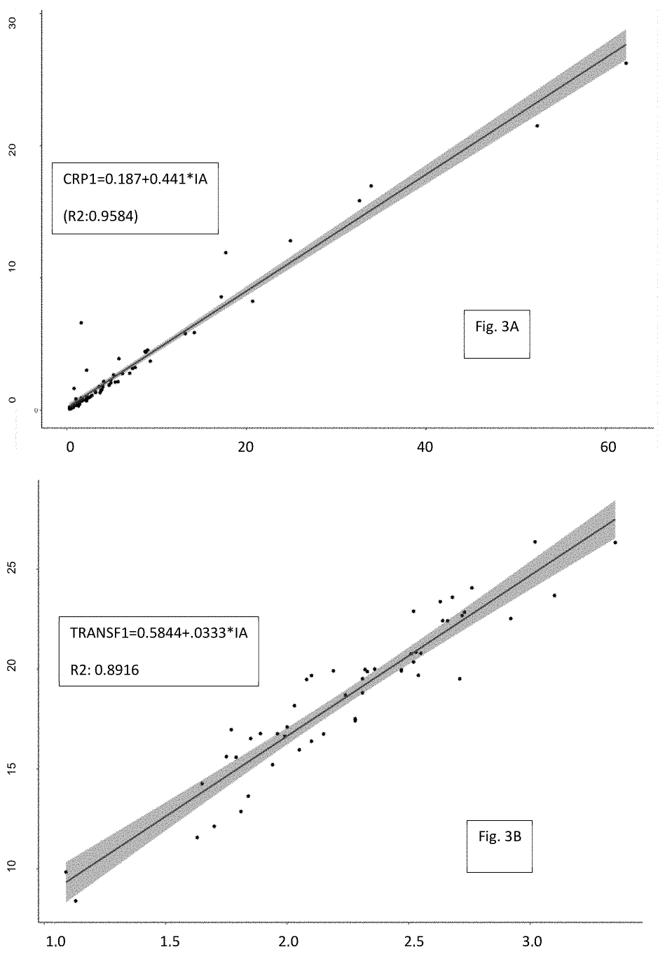

FIG. 3 diagrammatically represents the comparison of the quantification, in 95 plasma samples, of two serum proteins: C-reactive protein (CRP) (FIG. 3A) and serotransferrin (TRANSF) (FIG. 3B). For each of the proteins, the result of the quantification by clinically validated immunoassay is expressed on the x axis (in mg/L for CRP and g/L for TRANSF) and the result of the quantification by mass spectrometry on samples prepared using a process according to the invention is expressed on the y axis (arbitrary units).

The present invention will be better understood on reading the following examples which are given to illustrate the invention and not to limit its scope.

### EXAMPLE

### Example 1: Preparation of a Peptide Sample from a Dried Blood Sample (DBS) and Comparative Analysis by LC-MS

The efficiency of the process according to the invention was evaluated by detecting and quantifying 26 proteins by LC-MS, by comparing the analysis of a DBS sample treated using a process according to the invention (“DBS-RPW”) and, respectively, the analysis of a plasma sample treated using a process according to the state of the art (“plasma”) with an analysis carried out directly from a DBS sample (“standard DBS”). The steps of the process according to the invention are represented diagrammatically in FIG. 1A, the steps of the process for preparing a sample from plasma and according to a process of the state of the art are represented diagrammatically in FIG. 1B. The experiment was carried out in duplicate and each LC-MS analysis was carried out twice. The 26 proteins are the following: A1BG, A2MG, ANT3, Apo A1, Apo A2, Apo B100, Apo C2, Apo E, B2M, CERU, CF_X, C2, C3, C4B, CRP, CysC, FIBA, HPT, FIBB, IGFB3, PLMN, ORM, RET4, TRANSF, TTHY, ALBU.

The process of preparation according to the invention from a DBS sample according to the invention “DBS-RPW” is carried out as follows. To extract the proteins, a “DBS punch” is effected by punching the solid support (type 226 blotting paper) containing the sample. Said punch is transferred into a 96-well plate, then the proteins are extracted by adding 200 μL 50 mM ammonium bicarbonate, then by stirring for 30 minutes using a benchtop mixer at 350 rpm on an Eppendorf® Thermomixer Compact apparatus. The sample is then denatured by addition of 200 μL 8 M urea and stirring again for 10 minutes. The disulfide bonds of the proteins in the sample are then reduced by addition of 21 μL 200 mM DTT in 1 M Tris pH 8.5, and of 12 μL 1 M Tris pH 8.5, and stirring for one hour at 37° C. with stirring at 350 rpm on an Eppendorf® Thermomixer Compact. The released cysteines are then alkylated by addition of 18 μL 1 M IAA, 6 μL 1 M Tris pH 10, and stirring again for 30 minutes at 37° C. The alkylation step is stopped by addition of 20 μL 200 mM DTT. The sample is then acidified by the addition of 10 μL formic acid before transferring the 210 μL of supernatant into two new wells.

The step of cleaning of the sample is then carried out on RP-W® cartridges marketed by Agilent. The cartridges are washed and conditioned with 100 μL of an acetonitrile (70%)/TFA (0.1%)/water (29.9%) solution at 300 μL/min, and are brought into balance with 50 μL of a 0.1% formic acid solution at 10 μL/min. The sample is loaded onto an RP-W cartridge (Cat #G5496-60086) at 5 μL/min. The RP-W phase contained in the cartridge is washed with 50 μL of a 0.1% TFA solution at 10 μL/min and then the cleaned sample is eluted from the cartridge with 20 μL of an acetonitrile (70%)/formic acid (0.1%)/water (29.9%) solution at 5 μL/min. The elution is evaporated to dryness (Speedvac™). The dry sample is resuspended in 37.4 μL 20 mM Tris pH 8.5; 5.6 μg trypsin/LysC is added in order to carry out the trypsin digestion. This reaction lasts 14 hours at 37° C. with stirring (350 rpm on an Eppendorf® Thermomixer Compact). The digestion reaction is stopped by addition of 3 μL formic acid. The 96-well plate is sealed with film and the samples are ready to be injected into the LC-MS system.

The preparation of the “DBS standard” samples is carried out as follows: one or two drops of capillary blood, obtained after pricking the fingertip with a lancet, are deposited on each DBS. It is also possible to deposit 70 μL venous whole blood collected in a tube on a DBS using a pipette. After deposition, the cards are left to dry for 2 hours at ambient temperature. They are placed in an individual plastic bag and can be stored, depending on the use, at ambient temperature, at 4° C. or frozen (−20° C. or −80° C.). Before analysis, the cards are returned to ambient temperature. A punch with a diameter of 6 mm is then removed for each spot and transferred into a 2 ml Eppendorf LoBind tube.

The preparation of the “plasma” samples is carried out as follows: the process is represented diagrammatically in FIG. 1B, it represents a typical process known in the state of the art and comprises the following steps: from 2 μL plasma that is liquid or deposited on a “Whatman 903 paper” or “Ahlstrom FN/226 paper” support, the proteins are denatured and reduced, then undergo an alkylation, an enzymatic digestion (in the presence of trypsin/LysC for 14 h at 37° C.), then a step of cleaning of the obtained peptides carried out on a column of the ZORBAX Eclipse Plus C18 type, followed by analysis by LC-MS.

The results are illustrated in FIG. 2. A positive value is observed for the majority of the proteins. This indicates a larger detectable quantity (better sensitivity) in comparison with the standard process, and a good correspondence between standard plasma and DBS RPW values is also noted.

As a whole, the mass spectrometry intensities found after treating the DBS sample using a process according to the invention (DBS RPW) are 19 times more intense than those obtained directly after standard DBS. These intensities correspond, for the majority of the proteins, to those obtained on a plasma sample prepared using a standard process.

To validate the process for preparing a peptide sample according to the invention and to define its performance, the following study was conducted: ten independent samples taken from patients on DBS (DBS-RPW process according to the invention) were used for the study. For each patient, two DBS punches were analysed independently and in duplicate. The coefficients of variation (CV) of 91 peptides, corresponding to 76 proteins, thus measured twice independently and in duplicate, were calculated. This makes it possible to estimate the variability of the whole process for analysing the DBSs (pre-analytical and analytical). The median of the CVs obtained over all of the peptides is 9.3%. The CVs range from 2 to 52%. By comparison, during the same study, the median of all of the CVs obtained, this time on the plasma taken from the patients at the same time as the DBS, was 6.7% with the CVs ranging from 2 to 48%. To validate the clinical relevance of the measures, a correlation between the values obtained in the plasma (those which are thus used for clinical medicine) and those obtained in the DBSs was calculated. 35 peptides show a very strong correlation between “standard plasma” and “DBS-RPW” (coefficient of correlation >0.8), 32 show an intermediate correlation (between 0.6 and 0.8) and 24 show a weak correlation (<0.6).

The plasmas of 95 different patients, recruited in chronological order and without a particular pathology, were analysed, on the one hand, by mass spectrometry on samples prepared using a process according to the invention and in parallel by immunoassay, on a COBAS 6000 machine (Roche Diagnostic) in the Biochemistry department of Montpellier hospital. The results obtained are presented in FIG. 3, with analysis of CRP (FIG. 3A) and of serotransferrin (FIG. 3B) respectively. The results obtained by the two methods were compared using the R software. The comparison shows a statistically significant correlation for each of the two proteins quantified.

The results thus obtained therefore make it possible to validate the clinical relevance of the analysis process comprising a process for preparing a peptide sample according to the invention.

1. A process for the in vitro preparation of a peptide sample from a biological sample, comprising the following successive steps:
a) denaturation of the proteins present in said sample;
b) reduction and alkylation of the proteins resulting from step a);
c) cleaning of the proteins resulting from step b) by reversed-phase chromatography on a solid polymeric support, said solid support comprising at least one polystyrene-divinylbenzene polymer; and
d) digestion of the proteins resulting from step c) by a protease.

2. The process according to claim 1, characterized in that said biological sample is a blood sample in liquid form selected from the group consisting of: whole blood, serum and plasma.

3. The process according to claim 1, characterized in that said biological sample is a blood sample selected from the group consisting of: whole blood, serum and plasma, in that said sample is in solid or dried form, and in that said process comprises, prior to the step of denaturation of the proteins, a step of extraction of said proteins from said sample in solid or dried form.

4. The process according to claim 3, characterized in that said blood sample in solid or dried form is a sample of the DBS (Dried Blood Spot) type deposited on a collection paper, preferably a paper of the blotting paper type.

5. The process according to claim 1, characterized in that the step of digestion of the proteins by a protease is carried out in the presence of trypsin/endoproteinase Lys-C with a quantity of enzyme/quantity of substrate protein ratio comprised between 1/10 and 1/200, for a duration greater than or equal to 2 hours.

6. The process according to claim 1, wherein the DBS-type sample is treated during step d) in the presence of trypsin/endoproteinase Lys-C with a quantity of enzyme/quantity of substrate protein ratio comprised between 1/50 and 1/100, for a duration greater than or equal to 2 hours.

7. A process for detecting or quantifying proteins in a biological sample, comprising a process for the in vitro preparation of a peptide sample, according to claim 1, followed by a step of detection or quantification of at least one protein by means of an analysis technique, preferably by means of mass spectrometry, preferably the Liquid Chromatography Mass Spectrometry (LC-MS) technique.

8. The process according to claim 7, characterized in that said at least one protein is selected from the following proteins: afamin, alpha-1-antichymotrypsin, alpha-1B-glycoprotein (A1BG), alpha-1-acid glycoprotein, albumin (ALBU), alpha-2-HS-glycoprotein, alpha-2-macroglobulin (A2MG), antithrombin-3 (ANT3), apolipoprotein B100 (Apo B100), apolipoprotein C2 (Apo C2), apolipoprotein D, apolipoprotein E (Apo E), apolipoprotein M, apolipoprotein (a), apolipoprotein al (Apo A1), apolipoprotein A2 (Apo A2), apolipoprotein a4, beta-2-glycoprotein 1, beta-2-microglobulin (B2M), beta-Ala-His-dipeptidase, C4b-binding protein (alpha chain), CD5 antigen-like, cDNA-FLJ53327, ceruloplasmin (CERU), cholinesterase, clusterin, coagulation factor X (CF-X), coagulation factor XI, coagulation factor XII, complement C1q subcomponent subunit B, subunit C of complement C1q, complement C1r subcomponent, complement C1s subcomponent, complement C2 (C2), complement C3 (C3), complement C4B (C4B), complement C5, complement component C8 (beta chain), complement component C9, complement factor B, complement factor D, complement factor I, cystatin C (CysC), corticoid-binding globulin, C-reactive protein (CRP), fibrinogen (alpha chain) (FIBA), fibrinogen (beta chain) (FIBB), fibronectin, fibulin-1, gelsolin, haptoglobin, haemoglobin subunit alpha, haemopexin, heparin cofactor 2, the acid-labile subunit of insulin-like growth factor binding protein, insulin-like growth factor binding protein 3, haptoglobin (HPT), inter-alpha-trypsin inhibitor heavy chain H1, inter-alpha-trypsin inhibitor heavy chain H2, insulin-like growth factor binding protein 3 (IGFB3), lipopolysaccharide-binding protein, lumican, neuropilin-2, orosomucoid (ORM), PEDF, plasminogen (PLMN), protein AMBP, prothrombin, retinol-binding protein 4, serotransferrin (TRANSF), serum amyloid A4 protein, thyroxine-binding globulin, transthyretin (prealbumin) (TTHY), vasorin, vitamin D-binding protein, vitamin K-dependent protein C, vitamin K-dependent protein S, retinol-binding protein 4 (RET4).

9. A use of a process according to claim 1 for the preparation of a peptide sample for the detection or monitoring of the progression of a condition selected from:
neurodegeneration, in particular Alzheimer's disease,
a neurological or psychiatric disease, and in particular multiple sclerosis and autism,
a state of deficiency, infection, inflammation or malnutrition, and
a chronic or progressive disease, in particular cancer, hepatitis or a metabolic disease.
